(19) United States
(12) Patent Application Publication
(10) Pub. No.: US 2009/0285906 A1
(43) Pub. Date: Nov. 19, 2009

(54) Preparation Made From Diptera Larvae For The Treatment Of Wounds

(75) Inventors: Jochen D'Haese, Velbert (DE); Heinz Mehlhorn, Neuss (DE); Thomas Ruzicka, Dusseldorf (DE); Jurgen Schmidt, Dusseldorf (DE); Helger Stege, Freiburg (DE)

Correspondence Address: RANKIN, HILL & CLARK LLP, 23755 Lorain Road - Suite 200, North Olmsted, OH, 44070-2224, US

(73) Assignee: ALPHA-BIOCARE GMBH, Dusseldorf (DE)

(21) Appl. No.: 11/722,035

(22) PCT Filed: Dec. 21, 2004
(86) PCT No.: PCT/EP2004/053624

§ 371 Date: Jul. 12, 2007

(51) Int. Cl. A61K 35/64; A61P 17/02

(52) U.S. Cl. 424/538

(57) ABSTRACT

The invention relates to obtaining and to the topical use of homogenates, extracts and constituents therefrom (e.g. enzymes, lipids, low-molecular proportions) from pupae of diptera, in particular pupae of flies (e.g., the genera Sarcophaga, Musca, Lucilia, Phormia, Calliphora) for the treatment of open or poorly healing wounds of any origin.

The invention relates to obtaining and to the topical use of preparations such as homogenates, extracts and constituents therefrom (e.g. enzymes, lipids, low-molecular proportions) from pupae of diptera, in particular pupae of flies (e.g., the genera Sarcophaga, Musca, Lucilia, Phormia, Calliphora) for the treatment of open or poorly healing wounds of any origin.

A large number of factors in a cascade of processes play a decisive role in wound healing in humans and animals. The immediate course can be divided into three main phases of different lengths, independent from the type of wounds (chronic/acute). It begins with the so-called exsudative phase, followed by the proliferative and finally by the reparative phase.

In the exsudative phase, which is clinically characterised by a wound oedema and traumatic pain, reactions having vasoconstrictive and haemostaseogical effects are in the foreground. Vascular defects that have developed are closed by thrombocytes. The processes taking their course attract macrophages, neutrophile granulocytes and lymphocytes, whereby phagocytosis processes are initiated which in the end start the debridement of the tissue.

After the removal of cell debris, the immigration of fibroblasts and vascular endothelial cells begins the proliferative phase of the wound healing. Apart from the growth of the cell mass caused by the immigration, there is also an increased release of cytokines and also of growth factors, which in turn promotes cell proliferation and also further neovascularisation. During the concurrent modification of the matrix in this phase (conversion of collagen I into type F), a well-capillarised granulation tissue develops in the end, which includes numerous macrophages, fibroblasts and also mast cells. This phase is followed by the epithelisation and reparative phase. This is characterised by an immigration of peripheral keratinocytes into the wound and its wound contraction. While the capillary density is reduced, the collagen content increases even further, which is of great importance for the strength of the scar.

However, if the flow of complex processes of these three wound healing phases is impaired, the consequence may be a delay in the wound healing, which in some cases may be considerable. If this process is extended to 6-8 weeks, this is considered a chronic wound healing disorder. Such circumstances often arise as a consequence of various diseases (e.g. as a consequence of immunodepression and diabetes mellitus, as a result of a varicosis or certain superinfections, etc.). Measures for improving the wound healing are then aimed at accelerating the processes in the three phases of wound healing mentioned. Thus, one of the primary causes of the delayed wound healing—the deficient formation of granulation tissue—must especially be eliminated, which is caused either by reduced endogenous debridement of the wound or by excess formation of cell detritus.

Surgical procedures such as a curettage or wound excision are suitable as measures for cleaning necrotically or fibrinously covered acute or chronic wounds. However, new traumas are caused by these methods which can lead to another delay in wound healing, in particular in chronic wounds. In the case of infected wounds, these measures can not be carried out at all, or only with an antibiotic prophylaxis. Another problem is the painfulness of the surgery, which can only be carried out under a local or general anaesthesia. A postoperative immobilisation is required in the case of more comprehensive debriding surgical measures. This is often particularly undesirable in the case of elderly patients who suffer from numerous other diseases. A semi-surgical method that requires a certain technical understanding, also on the part of the patients, is vacuum sealing. Here, the wounds are occlusively covered while being permanently exposed to suction, and the covering layers are continuously removed by the negative pressure. This method is very effective but, as a rule, also requires immobilization. Purely pharmacological methods are based on the use of proteolytic enzymes. Most of the substances on the market are either enzymes from protozoa, or these products are of bovine origin. In practical application, however, the efficacy is often found to be low, either because the concentration has been selected too low, or the half-life of the products is too short, or because the target substances do not correspond with the host range of the enzymes. It must be remarked that due to their low efficacy or because of lack of proven efficacy, a number of commercially available products are not offered on the market anymore.

As another measure for the improvement of wound healing, the use of living fly maggots of the species Lucilia sericata is known.

This mode of therapy, wherein externally cleaned maggots are brought onto the wounds, has been known for centuries from folk medicine, The maggots (larvae) of the flies feed on necrotic tissue, but not on healthy tissue, and also penetrate into deeper layers. The larvae of Lucilia sericata do not bite or gnaw off the necrotic tissue, but apparently lyse the necrotic tissue by means of substances secreted per os and then imbibe the cell pulp. For they do not possess any mouthparts, because the two mouth hooks serve the purpose of attaching them to the skin and can cause pain in the process.

With regard to the routine use of these living maggots in toto in wound healing, there are problems in three areas. The first problem, and certainly not the least important, is an ethical one. Thus, many people are revolted by the crawling maggots, which, in addition, can indeed also cause considerable pain when clinging to the wound with their mouth hooks. Furthermore, it is difficult to breed living fly maggots that are free from potential pathogens such as bacteria or fungi in order to avoid microbial infections of the patient. The fly maggots must also be kept sterile also during transport from the laboratory to the patient. Another problem that is just as important stems from the logistics. Fly maggots cannot be “arrested” in this stage, but continue to grow and pupate after a time. Therefore, it is very difficult to have appropriate larvae of a suitable size ready for use when an appropriate patient arrives in the hospital and to keep them ready for each new application.

During the efforts for developing new and more effective medicaments for the treatment of chronic and acute wounds in humans and animals, it has now been found, surprisingly, that the preparations according to the invention, which contain the ingredients of pupae and diptera (in particular the pupae of flies of the genera Sarcophaga, Musca, Calliphora, Lucilia, Phormia), can eliminate the disadvantages mentioned of the current therapeutic measures in wound healing.

Moreover, the use of extracts from fly larvae or fly maggots was known from DE 10149153 A1, DE 10138303 A1 and DE 19901134 A1. However, it was not to be expected that pupae extracts would possess the same effect as maggot extracts, since pupae do not feed anymore, that is, they actually do not require lysing enzymes. Therefore, it is surprising that the extract obtained from pupae has any effect at all. In addition, pupae extracts have numerous advantages over fly maggot extracts. Thus, the content of the pupae can be obtained completely cleanly, because the puparium is tightly sealed towards the outside and because the surface of the pupae can be sterilised. Moreover, the pupal stage can be maintained over weeks or even months by cold storage, which is much easier than the use of living maggots, since their development cannot be halted. Further advantages of the use of pupae versus fly maggots are mentioned in the description. Thus, the use of fly maggots leads to pain for the patients, and faeces of the fly maggots may soil the wounds and lead to bad odour. Furthermore, fly maggots lead to the patients feeling revolted and thus have a very low patient compliance.

The extract from diptera pupae according to the invention therefore constitute a significant improvement of the currently practiced therapy with living maggots of flies or the use of extracts thereof. By using the ingredients of pupae, it is possible to clean the surface most intensively and to sterilise it, to clean up the isolates obtained, filter them sterilely, to lyophilise them, to store them, and then to use them only when needed—and, in addition, in an exact dosage. As such a ready-made preparation, the ingredients thus prepared offer continuous possibilities for intervention that are not dependent upon the respective development stage of living maggots.

Thus, the invention relates to preparations having a wound healing action that can be obtained from the content of diptera, and in particular from the inside of fly pupae.

Another aspect relates to ingredients with a wound healing action that can be obtained from pupae, in particular of representatives of the genera Sarcophaga, Lucilia, Calliphora, Musca, Phormia or from a mixture of these species.

Suitable species are, for example, from the genus Sarcophaga S. camaria, from the genus Lucilia: L. sericata, from Musca: M. domestica, Phormia: P. regina, as well as from the genus Calliphora: C. erythrocephala. These species, which are wide-spread outdoors, and which are, furthermore, kept in many scientific institutions worldwide, can thus be bred easily and in large quantities as defined material for the extraction processes concerned.

In particular, the invention relates to extracts having a wound healing action that can be obtained from the insides of fly pupae as soon as their cocoon is formed.

The invention also relates to a method for producing suitable extracts with wound-healing action, characterized by the puparium having first been cleaned externally and liberated from potential pathogens. Then, the invention also relates to a method for obtaining extracts having a wound-healing action characterised by removal of the insides of the pupa, wherein the homogenate obtained can be separated into a water-soluble and a water-insoluble fraction. The soluble parts are kept.

Homogenisation can be carried out by adding chemicals, by mechanical homogenisation or by using ultrasound, The separation of soluble and undissolved parts can be carried out, for example, by filtration and/or centrifugation. The entire method is carried out with cooling for the purpose of better preserving the active substances in the isolate. Then, the dissolved homogenisation constituents are stored by freezing or freeze-drying. In addition, other known substances, e.g. buffers, salts, antioxidants or microbicidal substances, etc.) can be added for stabilising the constituents of the preparations (e.g. proteins, enzymes, proenzymes, lipids, low-molecular parts, etc.).

The preparations according to the invention are produced by larvae of the diptera mentioned being bred on suitable substrates (e.g. horse meat) and then put on wood chips, where the maggots can pupate. The fly pupae are collected, externally cleaned and used.

The preparations according to the invention include, in general, any and all preparations that are obtainable from diptera pupae, including homogenates, extracts or constituents therefrom, which are suitable for wound treatment.

The, in particular aqueous, extracts or isolates obtained are stored in suitable carrier media, for example physiological saline solutions, sterile electrolyte solutions, albumin solutions, oils or fats, prior to processing.

The invention also relates to the preparations according to the invention for use as medicaments, or medicaments obtainable from or consisting of the preparations according to the invention.

The invention further relates to the use of the preparations according to the invention for producing a medicament for wound treatment, in particular for topical use.

The invention also relates to medicaments, characterised by an active content of the extracts according to the invention or constituents thereof, together with a pharmaceutically suitable, physiologically compatible carrier substance, additive and/or other active or auxiliary substance. Because of the pharmacological properties, the inventive preparations or medicaments are suitable in particular for the therapy of superficial or deep chronic and acute wounds of any genesis.

The term “chronic and acute wounds of any genesis” is understood to be, for example, wounds such as surgical wounds that are supposed to heal intentionally or unintentionally per secundam, cut injuries, stab injuries, abrasions, bite injuries or shot injuries, as well as other wounds that cannot be treated per primam by means of a surgical suture or a primary wound closure. In addition, the term acute wounds also denotes all wounds which cannot heal per primam due to a microbial infection, and all wounds whose manifestation is 4 weeks and less. Chronic wounds are all injuries that are accompanied by the break-up of the integrity of the epithelium and are manifest for more than 4 weeks. In particular, poorly healing wounds based on a diabetes mellitus, a varicosis or venous thrombosis, a rheumatic disorder, an occlusive arterial disease, a disease of the lymphatic vessels, haematological diseases and during or after infections of wounds are meant with this term.

The invention also relates to a method for producing a medicament characterised by the extracts or constituents thereof having wound healing activity being brought into a suitable form of administration with a pharmaceutically suitable and physiologically compatible carrier, and, if necessary, further suitable active substances, additives or auxiliary substances.

Application of the medicaments according to the invention is usually done topically. Suitable pharmaceutical compositions for topical use on the skin are at hand, preferably, as an ointment, solution, suspension, cream, powder, liposomal or oleosomal formulations, gel, lotion, paste, spray, aerosol or oil. Vaseline, lanolin, polyethylene glycols, alcohols and combinations of two or more of these substances may be used as carriers. The preparations according to the invention, in particular in the form of extracts or enzyme isolates, are generally present in a concentration of 0.1% by wt to 100% by wt of the galenical composition, preferably of 1.0% by wt to 60% by wt.

Transdermal administration of the preparations or medicaments according to the invention is also possible. Suitable pharmaceutical compositions for transdermal uses can be at hand as individual plasters that are suitable for long-term close contact with the epidermis of the patient. Such plasters suitably contain the preparations according to the invention, in particular the extracts or isolates, in a aqueous solution that, if necessary, is buffered, dissolved and/or dispersed in an adhesive agent, or dispersed in a polymer. A suitable concentration of the active substance is from about 0.1% by wt to 75% by wt, preferably from 1% by wt to 70% by wt. As a special option, the active substance can be released by electrotransport or iontophoresis, as described, for example, in Pharmaceutical Research, 2: 318 (1986).

Moreover, the preparations according to the invention, in particular extracts or enzymes or enzyme isolates from these extracts can also be applied to the wound by means of wound dressings made from gauze, alginates, hydrocolloidal materials, foamed substances and/or silicone dressings that were coated, impregnated or treated with these extracts, enzymes or enzyme isolates, and are thus capable of releasing the active substances into or onto the wound or wound surface.

Suitable solid or galenical forms of preparation are, for example, granulates, powders, dragées, tablets, (micro) capsules, suppositories, syrups, juices, solutions, suspensions, emulsions, drops or injectable solutions as well as preparations with protracted release of active substances, during the production of which commonly used auxiliary or carrier substances such as disintegrants, binding agents, coating agents, swelling agents, lubricants, flavouring substances, sweetening agents and solubilizers are used. Mention is made of magnesium carbonate, titanium oxide, lactose, mannitol and other sugars, talcum, lactoprotein, gelatine, starch, cellulose and its derivatives, animal and vegetable oils such as liver oil, sunflower oil, peanut oil, or sesame oil, polyethylene glycol and solvents such as sterile water and mono- and polyhydroxy alcohols, such as glycerine as frequently used auxiliary substances.

Furthermore, the preparations according to the invention, in particular extracts, can be used in galenical compositions that contain the active substances, e.g. enzymes, in an inactive form, and which are then applied into or onto the wound, and are activated by adding specific substances.

A particularly preferred preparation according to the invention is obtained by producing an aqueous extract and subsequently lyophilising the aqueous extract, in particular the supernatant of the extract liberated from insoluble parts. A durable and dosable preparation is thus obtained.

The use of a powder or of lyophilisates that are dissolved with physiological solutions (e.g. 0.9% aqueous NaCl solutions) are examples. Given sufficient stability, the galenical preparation may also be a solution.

Application of the suitable pharmaceutical preparations is carried out following a mechanical wound cleaning. Mechanical wound cleaning is done, for example, by means of a bath or a rinsing of the wound with lactated Ringer's solution. After the application of the extracts according to the invention, the wound is optionally covered with hydrocolloidal wound dressings, or with self-adhesive surgical wrap. The change of the bandages with a renewed administration of the extracts or isolates according to the invention each time is carried out daily.

### EXAMPLE A

### Production of Extracts According to the Invention

Larvae of the species Musca domestica, Calliphora erythrocephala, Lucilia sericata, Phormia regina, and/or Sarcophaga camaria are harvested from fresh, superficially flamed and uncontaminated horse meat and stored in suitable containers for pupation. The pupae collected then are externally cleaned in several washing steps in sterilised isoosmotic saline solution. The pupae are then crushed and the contents are collected in a carrier medium on ice. The carrier medium may either be aqueous or hydrophobic. Then, the ingredients a homogenised. This is done in several steps by means of ultrasound or mechanical homogenisers, or by adding solvents. Attention is paid to a continuous cooling at 4° C. After a homogeneous liquid has been obtained, the extract is aseptically filtered through a sterile filter having a pore diameter of 0.2 μm. In the final process step, the extract is aliquoted and frozen in liquid nitrogen. Lasting storage is done at about −18° C. to −80° C.

### Example of use A: Wound Treatment in Patients

An 83-year old patient with a PTS developed a chronic ulcus cruris at a typical localisation. Despite intensive treatment over more than 12 weeks, including enzymatic wound therapeutics, anti-inflammatory, disinfecting measures and a sufficient compression therapy the ulcer could not be caused to heal. Even after 12 weeks, the wound exhibited distinct coverings of fibrinous, partially inflammatory origin. The isolates were applied onto the wound once daily in aqueous form and as a lyophilisate. Then, the wound was covered with a lipid gauze free of active substances and bound with two compression bandages. Within 7 days, a very pronounced granulation tissue showed itself, without remnants of a fibrinous covering. The isolates continued to be applied once daily. Within 35 days, a reduction of more than 60% of the wound surface occurred, and after 59 days, the ulcer was healed completely.

### EXAMPLE B

### Extract from a Fly Pupae Species

By and by, 1,500 fly maggots of the species Lucilia sericata were taken from the production cages and collected in the refrigerator until the number was reached. Then, they were mechanically homogenised and at first centrifuged with a refrigerated centrifuge for two hours. The supernatant was then additionally centrifuged for twelve hours with a Beckmann ultracentrifuge. The supernatant was then sterilised and deep-frozen at −80° C. in portions of 1 ml. Portions of 1 ml were used after defrosting.

The obtained extract, additionally, was lyophilised according to standard and can then be stored in the refrigerator in aliquots suitable for practical use (for immediate use) at +4° C. or lower temperatures.

### EXAMPLE C

### Extract from Two Fly Pupae Species

750 pupae each of the fly species Lucilia sericata and Sarcophaga carnaria were collected in the refrigerator. Then, every group was processed as described in example A. Storage of the extract was done individually for each species. For treatment, 0.5 ml of the extracts of each of the two pupae species were defrosted and mixed, then shaken vigorously and applied on the wound.

The obtained extracts, additionally, were lyophilised according to standard and can then be stored in the refrigerator in aliquots suitable for practical use (for immediate use) at +4° C. or lower temperatures,

Example of use B: Product from Pupae of one Fly Species

A 76-year old patient with a ulcus cruris that had been recurrent for years and serious chronically venous insufficiency presented herself. The present ulcer had existed for several months and had previously been treated with conventional measures of moist wound treatment. Clinically, a fibrinous ulcer covered slightly necrotically presented itself. Within the context of a attempt at healing, extracts from pupae of the genus Lucilia sericata (1 ml/2 cm²) were applied on the wound and covered with a lipid gauze free from active substances as well as dry compresses and a compression bandage. Initially, application was daily. After 14 days, a nearly complete removal of the fibrinous coverings and the induction of a granulation tissue could be observed. Subsequent to this phase, therapy was carried out every two days. Now, in addition to the application with extracts from Lucilia sericata pupae, the ulcer was covered with wet compresses or hydrocolloid-containing wound dressings . After a further four weeks, we observed an initial epithelisation. A completely healed ulcer was evident after 94 days of the wound treatment. The successful therapy can clearly be assigned to the action of the extracts from Lucilia sericata pupae, since the other measures in the case history did not lead to a successful treatment.

Example of use C: Product from Pupae of Two Fly Species

For months, a 64-year old female patient suffered from a chronic wound in the area of the left lower leg due to a recurrent vasculitis. Therapy with glucocorticoid steroids systemically had slowed the activity of the vascular inflammation but could not lead to an effective induction of granulation tissue. Daily surgical debridement was very painful for the patient and regularly exhibited recurrence of fibrinous coverings. Only after daily application of extracts (50% to 50% volume fraction) from pupae of the fly genera Lucilia sericata and Sarcophaga carnaria, a complete removal of the necrotic and fibrinous material that had covered the wound occurred within ten days, together with an incipient epithelisation. Thus, the extract from two genera of flies is also active. This offers potential for saving costs.

1. A preparation obtainable from diptera pupae.

2. The preparation of claim 1, obtainable from pupae of the genera of flies Musca, Calliphora, Phormia, Sarcophaga or Lucilia, or proportions of two or several species.

3. The preparation of claim 1, characterized in that it comes from the species Musca domestics, Calliphora erythrocephala, Sarcophaga canaria, Phormia regina, Lucilia sericata and/or Lucilia Caesar.

4. The preparation of claim 1 characterized in that it comes from the pupation stages of flies.

5. The preparation of claim 1, characterized in that whole pupae or parts of pupae of the genera Musca, Calliphora, Phormia, Sarcophaga and/or Lucilia are homogenized mechanically, by the addition of chemicals and/or by ultrasound.

6. The preparation of claim 5, characterized in that the homogenates are liberated from undissolved constituents, and that soluble constituents are preserved.

7. The preparation of claim 1, characterized in that the separation of insoluble constituents is carried out by centrifugation, ultracentrifugation, phase separation or filtration and that only the supernatant is used.

8. The preparation of claim 1 characterized in that a sterile filtration is carried out with a filter having a diameter of pores of 0.1 μm to 0.4 μm .

9. The preparation of claim 1, characterized in that agents for preservation, selected from the group consisting of microbicidal substances or antioxidising substances, or combinations thereof are added.

10. The preparation of claim 1, characterized by the addition of pharmaceutically suitable and physiologically compatible auxiliary substances.
